(19) United States
(12) Patent Application Publication
(10) Pub. No.: US 2005/0277896 A1
(43) Pub. Date: Dec. 15, 2005

(54) Device for securing injection needles

(76) Inventors: Walter Messerli, Zollikofen (CH); Eugen Bucher, Muehlethurnen (CH)

Correspondence Address: DORSEY & WHITNEY LLP;INTELLECTUAL PROPERTY DEPARTMENT, 50 SOUTH SIXTH STREET, MINNEAPOLIS, MN, 55402-1498, US

(21) Appl. No.: 11/136,337

(22) Filed: May 24, 2005

Related U.S. Application Data
(63) Continuation of application No. PCT/CH03/00753, filed on Nov. 17, 2003.

(30) Foreign Application Priority Data
Nov. 25, 2002 (CH) 1982/02

(51) Int. Cl. A61M 5/00

(52) U.S. Cl. 604/240

(57) ABSTRACT

A needle holding device for coupling to a needle-carrying needle support whereby the coupling establishes a hydraulic pressure-resistant, traction-resistant, and liquid-tight connection between the needle support and a liquid reservoir to which it is operably connected.

### CROSS-REFERENCE TO RELATED APPLICATION(S)

This application is a continuation of International Application No. PCT/CH2003/000753, filed on Nov. 17, 2003, which claims priority to Swiss Application No. 1982/02, filed on Nov. 25, 2002, the contents of which are incorporated herein in their entirety by reference.

### BACKGROUND

The present invention relates to devices and methods for securing, mounting, integrating, or attaching and, more particularly, to the use of such devices and methods in the art of injection devices. More particularly, the present invention relates to a device for securing needle supports as used in injection apparatus and methods.

Injection devices or injection apparatus for injecting fluid medications have been known for a long time. For example, see Swiss patent Application No. 1984/02, entitled “Device for Automatically Injecting an Active Agent.” They often possess a sleeve-shaped main element, in which are contained a fluid reservoir, in most cases in the form of a replaceable ampoule or a syringe, as well as an expelling device. Prior to the injecting of a fluid, an injection needle (in the following often referred to as simply the needle) is attached to the injection apparatus.

Cone-type needle supports for connecting an injection needle to a fluid reservoir are known, as described in the ISO 594-1 standard. In the known embodiments, however, it is disadvantageous that the cone is subjected to the hydraulic pressure of the fluid to be expelled, which pressure results from the cross-sectional narrowing between the fluid reservoir and the injection needle and the consequent increase of the flow velocity of the fluid to be expelled. From this arises the danger that the needle support, together with the needle, will separate from the ampoule during the injection process.

A needle support can detach from the ampoule not only through the hydraulic pressure, but also through inertial forces that can develop in an automatic inserting process, primarily in association with autoinjectors. This danger is especially great when the injection needle penetrates into little or no resistance, in this case into human or animal tissue. An inserting unit, in the case of autoinjectors usually spring-driven, is not sufficiently damped. Deceleration of the inserting unit, especially when it runs undamped into a stop, is so great that the needle support, together with the needle, may fly off in the manner of a missile. If the needle support becomes detached from its connection before or during the injection process, then the fluid is not completely injected and, in addition, there exists an increased risk of needle breakage. Further, the patient must clean the injection apparatus of the liquid that has escaped between the ampoule and the unsealed needle support, which is undesirable. Also imaginable is the case in which the needle support detaches from the ampoule during the removal of the needle from the tissue. Although at this point in time the injection has already concluded, this is very undesirable to the patient and possibly extremely painful.

In addition, the known solutions do not allow one to insert the liquid reservoir together with the preassembled needle, needle support, and associated protective needle cap (which may be referred to herein as the protective cap) into the injector, and to remove the protective cap only shortly before an injection is to be carried out, even though this would be advantageous.

### SUMMARY

The present invention is intended to address the above concerns. It involves the task of providing a device for securing or creating a liquid-tight, conical connection between the needle support and the fluid reservoir, which connection can withstand hydraulic pressure, acceleration forces, and tensional forces. Thus, among other things, the danger of an incomplete injection and the danger of a needle breakage are greatly reduced. In addition, it should be made possible to insert the liquid reservoir together with the preassembled needle, needle support, and associated protective needle cap into the injection apparatus.

In one embodiment, the present invention comprises a needle holding device for coupling to a needle-carrying needle support whereby the coupling establishes a hydraulic pressure-resistant, traction-resistant, and liquid-tight connection between the needle support and a liquid reservoir to which the needle support is operably connected.

In one embodiment, the present invention comprises a device for securing a needle support and injection needle connected to the support to a liquid reservoir, wherein the device comprises at least one needle holder.

Advantages achieved by embodiments of the present invention are that the needle holder ensures a hydraulic-pressure proof, tension-proof, and liquid-tight conical connection between the needle support, connected as one piece to the needle, and the liquid reservoir during an entire injection process up to and including the removal of the injection needle from a patient.

In some embodiments, the diameter of the needle holder is at least as large as the diameter of the collar of the protective cap and makes possible, as a further advantage, the inserting of the liquid reservoir together with the preassembled needle, needle support, and associated protective needle cap into an injector, which in particular may be an autoinjector of known design, and the taking off of the protective cap from the needle, which is inserted together with the liquid reservoir, only shortly before the injection to be carried out. One such process is described in the Swiss application entitled “Device for Temporarily Retaining a Protective Needle Cap of an Injection Apparatus”, (Application No. 1985/02).

### BRIEF DESCRIPTION OF THE DRAWINGS

FIG. 1 is an exploded view of an injection apparatus;

FIG. 2 depicts an embodiment of a needle holder according to the present invention;

FIG. 3 depicts the liquid reservoir inserted into the injection apparatus together with the preassembled needle, needle holder according to the invention, and associated protective needle cap; and

FIG. 4 depicts a further embodiment of a needle holder according to the present invention.

### DETAILED DESCRIPTION OF THE DRAWINGS

In the following description, the terms proximal and distal are used in the sense that is conventional in medicine, thus:
proximal=turned away from the patient, and
distal=turned toward the patient.

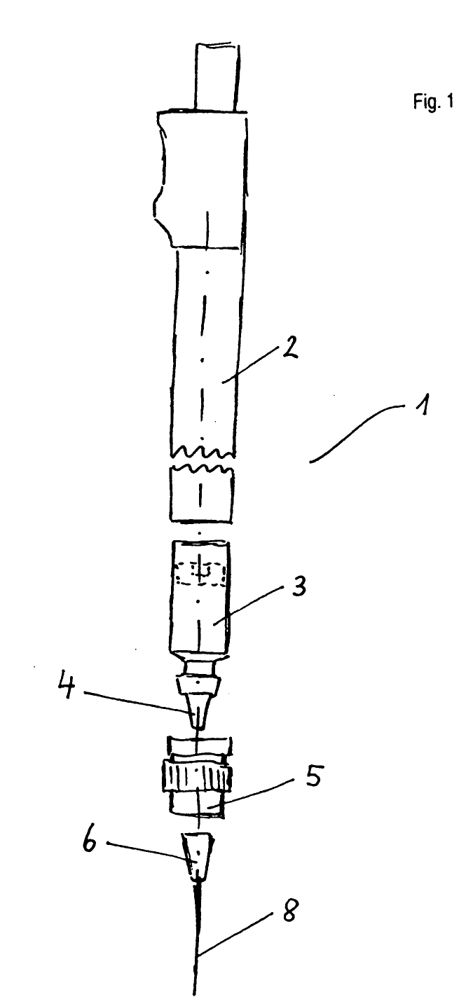

As shown in FIG. 1, a fluid reservoir (3), for example an ampoule that is prefilled or to be filled by the patient himself with a fluid, is pushed from the distal side into the housing part (2) of an injection apparatus (1), in which it is situated on the proximal side of a dosing or expelling mechanism of suitable or known design. In order to fix the inserted liquid reservoir (3) in the housing part (2), a nut (5) is screwed onto the distal end of the housing part (2). Through this means, the liquid reservoir (3) is reliably secured in the housing part (2). The correct and secure fitting of the liquid reservoir (3) significantly influences the dosing and expelling precision of the injection apparatus (1) and is the basis of a secure assembly of an injection needle (8) with one-piece connected needle support (6). Since the nut (5) displays an axial through-bore, a connection cone (4) of the liquid reservoir (3), when the nut (5) is screwed on, protrudes in such a way that an injection needle (8) with a one-piece connected needle support (6) is placed onto the distal end of the liquid reservoir (3) only shortly before the injection to be carried out.

The unsecured connection cone (4) is subject to the entire hydraulic pressure that develops during the expelling of the contents from the liquid reservoir (3). Further, in infrequent cases, it can happen that in the removal of the injection needle (8) from the tissue the frictional resistance of the injection needle in the tissue is greater than the friction of the conical connection of the needle support (6) to the liquid reservoir (3). Thus, the injection apparatus (1) is unintentionally separated from the needle support (6) before the injection needle (8) has been removed the tissue. The injection needle (8) together with a sleeve-shaped protective needle cap (21) (not shown in FIG. 1) can be placed onto the liquid reservoir (3) only after the latter has been inserted into the housing part (2) of the injection apparatus (1). In the patients' view, especially in the case of so-called individual doses, in which a new liquid reservoir (3) is needed for each injection, it would be desirable if the liquid reservoir (3) could be placed into injection apparatus (1) together with the associated injection needle (8).

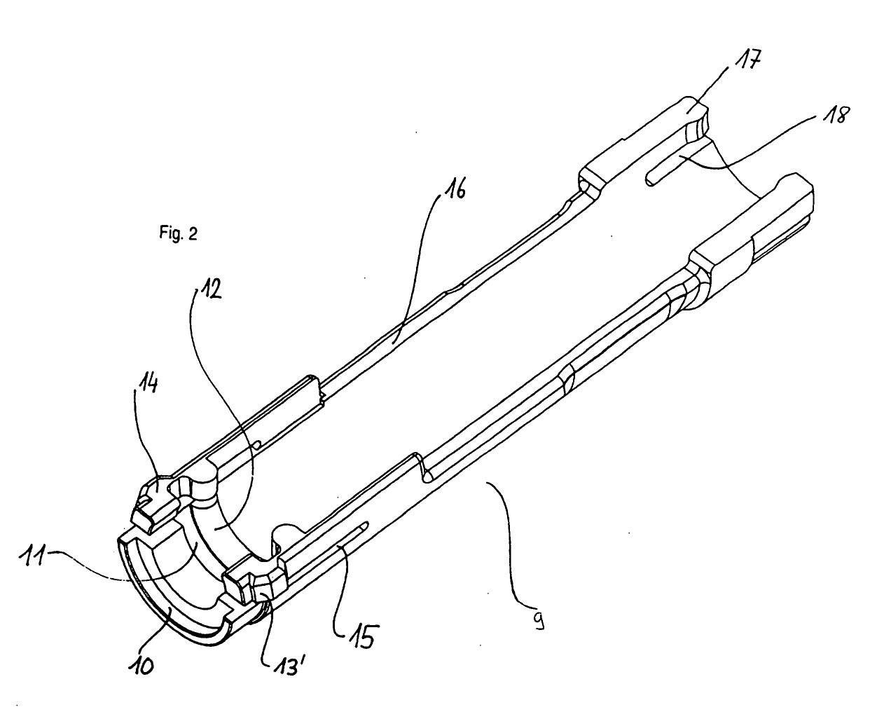

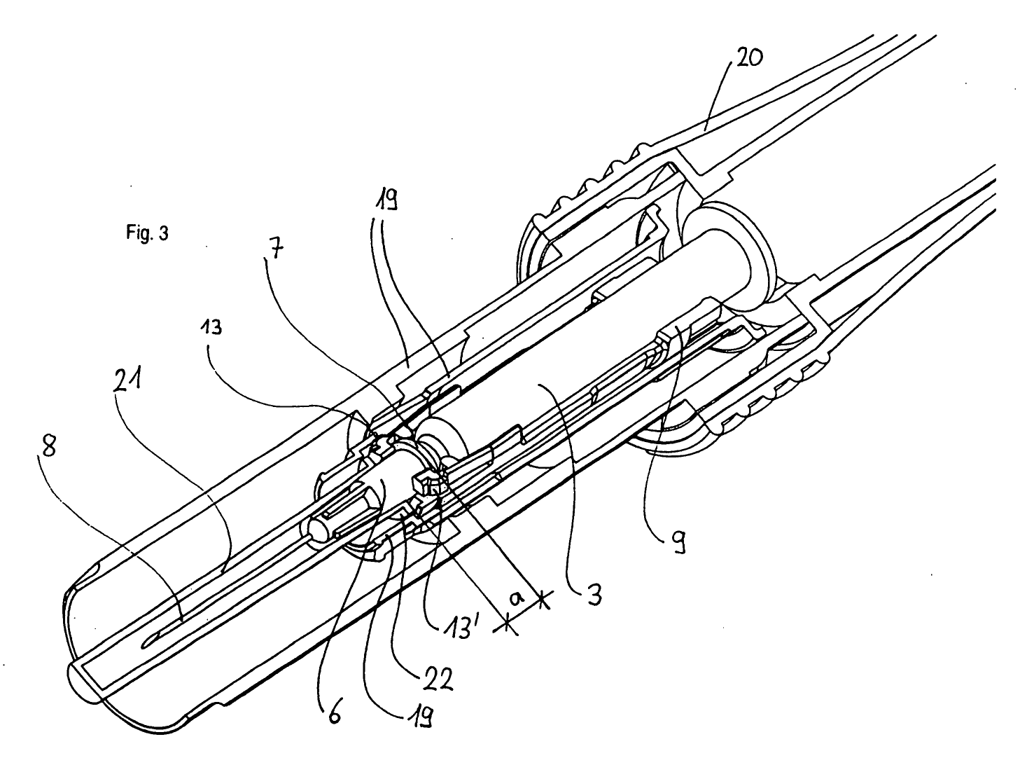

A needle holder (9) in the form of a half-shell, as shown in FIGS. 2 and 3, addresses the above-mentioned disadvantages. The needle holder (9), which is preferably made of plastic or other suitable material, is snapped onto the liquid reservoir (3), which, in some preferred embodiments, consists of a commercial syringe or ampoule for administration of single or multiple doses, and in particular contains a medication such as insulin, heparin, erythropeotin (EPO), or nutropin. In the region of a latch (17), which is situated at the proximal end of the needle support, the needle holder (9) according to the invention surrounds the liquid reservoir (3) around at least 180 degrees, but in some preferred embodiments, more. Through this means, the needle holder (9) is connected to the liquid reservoir (3), which at least in the region of the latch consists of a glass or plastic tube, preferably cylindrical. An elasticity slit (18) ensures that the snapping force of the latch (17), which in this embodiment has the form of a latch leg, is located in a region corresponding to the use. Also conceivable is the achieving of the necessary elasticity through the purposeful adjustment of the material cross section, especially in the context of a viscoplastic material such as, for example, soft PE.

The liquid reservoir (3) is usually provided on its cylindrical outer surface with information important to the patient, as for example the description of the liquid contained in the liquid reservoir (3), its expiration date, etc. The purpose of a groove-shaped cutout (16) is to ensure that this information is not unnecessarily covered up. Since the liquid reservoir (3) is rotationally-symnetrical at least in the region where it comes to rest in the needle holder (9), the user can rotate the liquid reservoir (3) in the needle holder (9) according to the invention in such a way that a maximum of information is visible to him.

A cylindrical collar (7) of the needle support (6) is axially secured in a groove (11) of the needle holder (9). An axial force provider (not shown), in some preferred embodiments, a compression spring, attached in a rear housing part (20), presses forward the liquid reservoir (3), inserted in a front housing part (19), together with the needle holder (9) and needle support (6). Both housing parts (19, 20) preferably consist of several individual parts, which are not described here in detail.

Both the front housing part (19) and the needle holder (9) display a support bevel (13) and (13′), respectively. The pressure force (not shown) presses the two support bevels (13) and (13′) onto each other. The wedge effect of the support bevels (13) and (13′) pressed onto each other produces, due to the force acting in the axial direction, a radial force component, which compresses locking elements, in this case holding legs (14) of the needle holder (9), in a radially inward manner, in order to connect the needle support (6) to the needle holder (9) in an interlocking manner. Through this means, the injection needle (8) with the one-piece connected needle support (6) is connected to the front housing part (6) via the needle holder (9) in an at least tension-proof manner. Through this connection, according to the invention, it is ensured that the hydraulic pressure no longer must be received exclusively by the connection cone (4). Through the axial spring force, not only is the needle support (6) connected to the front housing part (19) in an at least tension-proof manner, but also, in the connection cone (4) the contact pressure sometimes necessary for liquid sealing is ensured during the entire injection process.

Through elasticity slits (15) the holding legs (14) are very flexible, so that the axial force is sufficient to radially inwardly compress the holding legs (14) via the support bevels (13, 13′). In some embodiments, in the context of a viscoplastic material and an adapted deformation cross-section, it would be possible to eliminate the elasticity slits (15) or to reduce their size.

A rib (12) prevents faulty manipulations through axially displaced installation of the needle holder (9). If a cylindrical collar (22) of a sleeve-shaped protective needle cap (21), instead of the cylindrical collar (7) of the needle support (6), comes to rest in the groove (11), then the holding legs (14) are spread apart by the rib (12) in such way that the additional needle holder (9) can no longer be inserted into the front housing part (19). Through this means, the patient is alerted in time to a possible faulty installation of the needle holder and can correctly install the needle holder.

The needle holder (9) displays a diameter that is at least as large, but preferably slightly larger, than the collar (22) of the protective needle cap (21). Achieved thereby is that the liquid reservoir (3), together with the needle support (6), the injection needle (8), the associated protective needle cap (21), and the additional needle holder (9), can be inserted axially into the front housing part (19) and the needle protective cap (21) can be removed at any time. This advantageously facilitates patient use.

Depending on production processes and tolerances, not all protective needle caps (21) are equally tight-fitting. Resulting from this is a different distance (a) as shown in FIG. 3. In order to be able to correctly install the additional needle holder even in the case of a very short distance (a), a recess (10), in some embodiments, preferably cylindrical, releases the collar (22) of the protective needle cap (21).

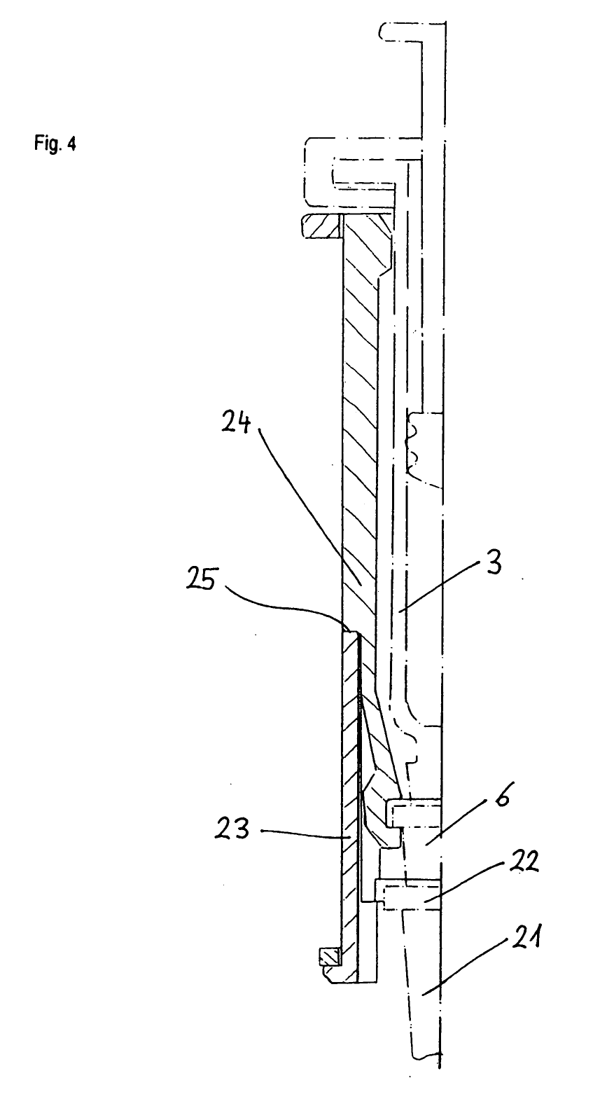

A further possible embodiment of the present invention comprises a tube-shaped needle holder (23) with a lateral cutout as shown in FIG. 4. In the lateral recess, a locking part (24) is inserted axially, in the distal direction of the injection needle. Through the fact that the locking part (24) is deflected inwardly by the tube-shaped needle holder (23) in the region of the needle support (6), the needle support (6) is held in an interlocking manner. So that the locking part (24) cannot be pushed too far in the direction of the injection needle (not shown), a step (25) limits the travel. In the case of an injector with automatic needle insertion, usually called autoinjectors, the tube-shaped needle holder (23) can simultaneously assume the function of a sliding sleeve that, aided, for example, by a spring element, carries out an inserting stroke. In this embodiment, the tube-shape needle holder (23) displays a diameter that is at least as large as the collar (22) of the protective needle cap (21).

While exemplary embodiments, including preferred embodiments, of the present invention have been described herein, it is contemplated that various modifications could be made without deviating from the spirit of the present invention. Accordingly, it is intended that the scope of the present invention be dictated by the appended claims.

1. A device for securing a needle support and a needle connected thereto to a liquid reservoir, comprising at least one needle holder.

2. The device according to claim 1, wherein the needle holder secures the needle support and injection needle in a liquid-tight manner.

3. The device according to claim 1, wherein the needle holder secures the needle support and injection needle in a pressure-proof manner.

4. The device according to claim 1, wherein the needle holder secures the needle support and injection needle in a tension-proof manner.

5. The device according to claim 1, wherein the needle holder has a diameter at least as large as a collar of a protective needle cap.

6. The device according to claim 1, wherein at least the needle holder, together with the liquid reservoir, the injection needle, and the needle support, can be pressed against a support bevel through an axial force.

7. The device according to claim 6, further comprising locking elements radially compressed by cooperating support bevels to connect the needle support and needle carried thereby to the liquid reservoir in an interlocking manner.

8. The device according to claim 1, wherein the liquid reservoir is connected to the needle support and injection needle by means of a conical connection.

9. The device according to claim 1, wherein the needle holder comprises at least one rib that, in the case of an axially displaced installation of the liquid reservoir, effects a deformation of the needle holder and thereby precludes an insertion into an injection apparatus.

10. The device according to claim 1, wherein the needle holder comprises at least one latch.

11. The device according to claim 10, wherein the needle holder snaps onto the liquid reservoir.

12. The device according to claim 1, wherein the needle holder, together with the needle support, injection needle and a needle cap are operably coupled to an injection apparatus.

13. The device according to claim 12, wherein the needle cap is removeable from the injection needle while coupled t.

14. The device according to claim 1, wherein the needle holder comprises at least one recess for receiving a collar of a needle cap.

15. A device for securing a needle support to a liquid reservoir, wherein an injection needle is carried by the needle support, and wherein the device comprises at least one needle holder.

16. A holder for receiving a container for containing a liquid to be dispensed, the container carrying a needle support carrying a needle, said holder adapted to be operably coupled to a device for injecting, said holder comprising: a body adapted to receive the container, the body having two ends and, adjacent to one end, a relieved region for receiving a portion of the needle support, a bevel structure for cooperating with a portion of the needle support, and locking elements for engaging a portion of the needle support.

17. The holder according to claim 16, wherein the locking elements comprise radially moveable legs.
